(19) United States
(12) Patent Application Publication
(10) Pub. No.: US 2020/0383906 A1
(43) Pub. Date: Dec. 10, 2020

(54) HORMONAL CONTRACEPTION USING A VAGINAL RING WHICH RELEASES ESTRIOL AND TRIMEGESTONE

(71) Applicant: Evestra, Inc., Schertz, TX (US)

(72) Inventors: Walter Elger, Berlin (DE); Klaus Nickisch, Berlin (DE)

(21) Appl. No.: 16/893,312

(22) Filed: Jun. 4, 2020

(30) Foreign Application Priority Data
Jun. 6, 2019 (DE) 102019115343.3

(51) Int. Cl. A61K 9/00; A61K 31/566; A61K 31/57; A61K 31/575; A61P 15/18

(52) U.S. Cl. CPC A61K 9/0036; A61K 31/566; A61P 15/18; A61K 31/575; A61K 31/57

(57) ABSTRACT

The invention relates to an intravaginal drug delivery device for hormonal contraception with prevention/reduction of intermenstrual bleeding and menopausal cycle disorders. The intravaginal drug delivery device includes estriol or a precursor of this compound and a progestogen or a precursor thereof.

### PRIORITY CLAIM

This application claims priority to German Patent Application No. 10 2019 115 343.3 filed on Jun. 6, 2019, entitled “Vaginalring für die hormonelle Kontrazeption” (“Vaginal Ring for Hormonal Contraception”), which is incorporated herein by reference.

### BACKGROUND OF THE INVENTION

1 Field of the Invention

The invention generally relates to a preparation for hormonal contraception with prevention/reduction of intermenstrual bleeding and menopausal cycle disorders. The method comprises the vaginal application of a combination of estriol as estrogenic component with trimegestone as progestogenic component using a Vaginal Ring (VR) to a potentially fertile women in an effective amount to inhibit ovulation.

### 2. DESCRIPTION OF THE RELEVANT ART

Estrogen is the most important female sex hormone. It is crucial for the function of the female reproductive system and for the secondary sexual characteristics. The three most important female endogenous estrogens are estrone, estradiol, and estriol. They differ in the strengths of their estrogenic hormone properties.

Estrogens are used as drugs, inter alia, for hormonal birth control and for hormone therapy during menopause. Estradiol is the dominant estrogen during the reproductive years. This applies to both serum level and estrogenic activity. During menopause, by contrast, estrone is the predominant circulating estrogen. During pregnancy, estriol is the dominant estrogen.

Estrogens play an important and mayor role in existing methods of hormonal contraception. Estrogens are needed for inhibition of follicle maturation and ovulation. Beyond inhibition of ovulation this treatment intends to replace the ovarian hormone secretion which in part or completely abolished by the suppression of follicle growth and ovulation. Subsequent absence of a corpus luteum goes along with the absence of a corpus luteum and its secretion of progesterone. This is substituted by the progestin in the estrogen-/progestin combination.

Combined oral contraceptives known from the state of the art consist of an estrogen-progestogen combination. The preparations are taken daily for a period of 21 days. This is followed by a hormone-free period of 7 days. Within the seven days, two or three days after ingestion of the last tablet the hormone withdrawal bleeding sets in.

Natural estrogens are pharmacokinetically deficient for an oral contraceptive. 17α-ethinyl estradiol (EE) is still the leading estrogenic substance in the combined hormonal contraception. EE is contained in the leading oral and—surprisingly—also in non-oral—contraceptive products. It is used in the contraceptive vaginal ring (e.g. the Nuvaring®) and contraceptive transdermal patches.

The main issue with EE arises from the fact that the liver is a target organ for estrogens. The spectrum of hepatic secretion activities that is affected by estrogens in the human liver includes synthesis of binding proteins CBG, SHBG, TBG, and several factors that are important for the physiology of blood clotting, angiotensinogen, lipoproteins, and an array of other factors. The strong hepatic estrogenicity of ethinyl estradiol (EE), especially its effects on hemostasis factors, may explain why contraceptives containing synthetic estrogen EE has been associated with the observed enhanced risk of thromboembolism in oral contraceptive users. Other undesirable side-effects of synthetic estrogens include fluid retention and an increase in blood pressure.

The aforementioned deficits of EE are of considerable significance and consequently there is a significant unmet medical need for estrogens that do not display these deficits and which can suitably be employed in contraceptive methods. Such estrogens would need to have the ability to reliably suppress follicle maturation and ovulation and effectively replace the endogenous ovarian secretion of estradiol.

A sufficient dose of EE is essential to suppress the FSH secretion of the pituitary and follicle growth in the ovary. Under borderline EE doses (≤20 μg EE/day, orally) follicle growth in the ovary is incompletely suppressed. Incompletely suppressed follicles may achieve maturity. A particular risk in this context is the 7- days pill-free interval of the (21 days on +7 days off scheme) 28 days' pill cycle. Preexisting follicles may reach maturity during and following pill withdrawal. The pills of the next cycle may no longer be able to prevent ovulation.

Unfortunately, non-oral treatment using EE, in an attempt to avoid the impact of the first liver passage, failed to reduce the known hepatic estrogenicity of this compound. This applies also to the well investigated health risks: Rather an increased risk of venous thromboembolism vs oral treatments was found with non-oral treatments using EE.

The oral activity of EE is due to its impaired metabolic inactivation. The oxidation of the 17OH- group of EE is chemically not possible. Human pharmacokinetic studies, using tritium-labeled EE, were performed comparing low and high doses of EE after intravenous and oral administration. High individual variation of bioavailability and terminal half-lives was recorded. A relatively low oral bioavailability of about 40% (first pass effect 60%) was seen.

Estradiol-17ß was also considered for oral contraception, using oral and various non-oral methods of administration. Oral studies in young women revealed a low, individually variable oral bioavailability on the order of 5% (4.9+/−5%) at two lower tested doses (2.0 and 4.0 mg). A high dose (8.0 mg) was incompletely taken up from the intestinal tract. This indicates a limitation to the dose of estradiol for use as a contraceptive.

Estradiol has also its limitations concerning non-oral treatment strategies. These limitations are likely due to endometrial metabolism of this hormone under progesterone dominance as in the luteal phase by an upregulation of the estradiol metabolizing enzyme 17ß-OHSD

Correspondingly E2-metabolism may prevail under the simultaneous administration with a progestin. The poor cycle control and breakthrough bleedings under oral and parenteral estradiol-/progestin combinations were attributed to this catabolism. The induced local estradiol deficiency in the endometrium explains the observed poor bleeding control.

A dose of 20 μg of EE is the lower limit with regard to arrest of follicle growth and bleeding control. The price for the oral estrogenic properties of EE are massive estrogenic effects in the liver. Effects include a larger number of estrogen modulated metabolic functions, lipid metabolism, factors of the hemostatic system, angiotensinogen secretion (electrolyte metabolism, blood pressure control), hormone binding proteins, and several other functions.

A dose reduction of EE does not prevent the undesired effects of EE on the human liver. Adverse effects are seen far below doses of EE which are needed for the safe inhibition of ovulation or the avoidance of irregular uterine bleedings. For example, a dose of 5 μg EE/day is still hepatically very active. The threshold of EE which produces these corresponding effects is probably not known. Dose reduction in an oral contraceptive is unlikely to solve the issue of strong hepatic estrogenicity of EE. The risk of dose reduction of EE is that there will be an accompanying loss of bleeding control and loss of contraceptive efficacy.

Non-oral administration of EE does not minimize its hepatic estrogenic effects. Pharmacokinetic and pharmacodynamical studies show that high hepatic estrogenicity of EE is not an effect of first liver passage, but is an inherent property of EE. EE has a long half-life in the circulation. Blood levels of EE determine the effects, irrespective of oral or non-oral administration. In a study, the effects of oral vs vaginal EE were studied in postmenopausal women receiving (a) 5 μg of EE orally and 20 μg EE vaginally or (b) 1 μg EE orally and 50 μg EE vaginally/day for 25 days, respectively. Antigonadotrophic effects were measured for the assessment of systemic estrogenic effects, SHBG and CBG and lipoproteins served as parameters for hepatic estrogenicity. Oral EE was four to five times more potent than vaginal EE. This showed that the intestinal tract was more efficient concerning the uptake of EE than the vagina. A selective relative reduction of hepatic effects using a parenteral administration was excluded.

Epidemiologic studies of cardiovascular risks of EE releasing contraceptive vaginal rings and transdermal products reveal no advantage concerning deep venous thromboembolism compared with oral administration. Rather higher blood levels of EE and risks compared even with second generation oral contraceptives were seen.

Estriol is secreted in large quantities by the human placenta. Products of the synthesis of estriol in the fetal adrenals and the fetal liver include DHEA (Dehydroepiandrosterone) and 16a OH-DHEA which is aromatized rendering estriol at the end of a complex steroidogenic assembly line in the placenta. In adult women, estriol may also arise from estradiol by 16α-hydroxylation and by as yet unidentified pathways

Compared with estradiol, estriol forms weaker and less stable binding with the estrogen receptor ERα and the estrogen receptor ERß. Estriol is about factor ten weaker in standard tests, e.g. the Allen Doisy test, in ovariectomized rodents at parenteral administration. This finding dominates the view on the estrogenic potency of these natural estrogens.

Treatment with an estriol bolus injection, intravenously, shows a dramatic elevation in target tissues due to receptor binding. The generated peak is reached faster and higher than that after an estradiol injection. An important difference appears the rapid decrease of tissue levels in case of estriol compared to long lasting elevations in case of estradiol.

Used in a broad spectrum of studies estriol was found to be a weak or “impeded” estrogen, in combination with estradiol, although some antiestrogenic activity was stated. The prior art studies thus do not favor estriol for a contraceptive strategy.

Vaginal ring projects have been described before. Currently, there are six intravaginal rings (IVRs) on the market. ESTRING and FEMRING are reservoir systems based on silicone, whereas PROGERING and FERTIRING are silicone-based matrix systems. GINORING is a reservoir system based on ethylene vinyl acetate (EVA) as core material and TPU (polyurethane) releasing two hormones, i.e., etonogestrel and ethinylestradiol. Devices of this type, however, do not provide pharmacologically acceptable and/or optimal release profiles.

NuvaRing® is a reservoir system built up of two different EVA co-polymer types. The core is based on an EVA with 28% VA and supersaturated with etonogestrel (ENG) (i.e., 11.7 mg), whereas the EE concentration is below its saturation solubility (i.e., 2.7 mg). An EVA with 9% vinyl acetate (VA) serves as the skin material, the skin thickness is around 110 μm according to the product description. Due to its reservoir design, the skin modulates the drug release. Both, EE and ENG are continuously released over 21 days with average daily in-vitro release rates of 15 μg and 120 μg during day 2 and day 20. However, the release rates may be increased on day 1 and decreased on day 21. On day 1, the so-called burst effect is likely to occur: Hormones, dissolved in the core material, gradually migrate into the skin according to their diffusion coefficient. Thereby, some hormones are located at/close to the IVR's surface and is immediately dissolved when placed into the dissolution medium. Thereby, increased release rates are observed. At late time points, the remaining hormone fraction in the core may be lowered yielding decreased release rates.

Improvement was sought by using other shapes or materials. A two-layered, one compartment vaginal ring made from silicon elastomer has been disclosed in European Patent No. 0 050 867, in which a silicone elastomer core is loaded with active substance surrounded by a non-loaded silicone elastomer layer which consists of two different compositions.

Another improvement was claimed in U.S. Pat. No. 4,292,965 which discloses a three-layered compartment ring. This ring is composed of an inert silicone elastomer core encircled by a medicated silicone layer and a non-medicated silicone outer layer.

Drug delivery systems for vaginal use, and in particular vaginal rings, prepared using polyethylene vinyl acetate (EVA) copolymers are also known in the art (see e.g., van Laarhoven et al. Journal of Pharmaceutics 232 (2002): 163-173).

European Patent No. 0 876 815 describes a one compartment vaginal ring composed of EVA copolymer core containing ethinyl estradiol and etonogestrel and a non-medicated EVA outer membrane, which controls the release rate of the active components. This device releases two or more active substances in an essentially constant ratio to one another over an extended period of time. This concept was further developed in U.S. Published Patent Application No. 2014/0302115 in which the basic concept remains the same, but by using certain EVA polymers, a higher stability at room temperature was reached.

Other concepts described include using drug delivery devices having three, instead of two layers, whereas at least two of the three layers consists of drugs, see e.g., U.S. Patent Application Publication Nos. 2012/0148655, 2014/0350488 and 2009/0081278. All these drug delivery devices release the active ingredients in an essentially constant release rate, where the rate of release is controlled via the outer layer.

Another approach to the above problem is described in U.S. Pat. Nos. 7,829,112; 7,833,545; 7,838,024 and 7,883,718 that describe drug delivery devices that have two or more unitary segments composed of a drug—permeable polymeric substance, where at least one of the segments has a pharmaceutically active agent. A special noteworthy property of the claimed devices is that they deliver the active pharmaceutical agents at a substantially zero-order rate and that the segments do not have a membrane.

In addition to single drug delivery, vaginal rings have been developed for the simultaneous release of more than one drug at one time. The vaginal rings described in U.S. Pat. Nos. 3,995,633 and 3,995,634 have separate reservoirs containing different active substances, wherein the reservoirs are arranged in holders. In U.S. Pat. No. 4,237,885 multi-reservoir devices are described in which spacers are used to divide a tube into portions. Each portion is filled with a different active ingredient in a silicone fluid. PCT Application No. WO 97/02015 discloses a two-compartment device wherein one compartment has a core, a medicated middle layer, and a non-medicated outer layer and a second compartment has a medicated core and a non-medicated outer layer.

A further improvement was disclosed in U.S. Pat. No. 5,989,581 for the simultaneous release of a progestin compound and an estrogen compound, reportedly in a fixed ratio over a prolonged period of time. This approach was further modified in PCT Application No. WO 2015/086491 which describes an intra-vaginal drug delivery system having a core covered by a skin. The core is composed of a first thermoplastic polymer and a first therapeutic agent, where the first therapeutic agent is dissolved in the first thermoplastic polymer. A skin surrounding the core is composed of a second thermoplastic polymer, wherein the first therapeutic agent is less permeable in the second thermoplastic polymer than in the first thermoplastic polymer. A second therapeutic agent is loaded in a portion of the skin.

However, like other devices, the ones disclosed in U.S. Pat. No. 5,989,581 and WO 2015/086491 suffer from their own inherent limitations. In general, the release per unit time of a drug is determined by the solubility of the active substance in the outer layer of polymeric material and by the diffusion coefficient of the active substance in the membrane. This is especially relevant if the two pharmaceutical ingredients have significantly different physicochemical properties in general and especially when it comes to different diffusion coefficients.

One approach to overcome the limitations of the low solubility of certain drugs in the polymer used as a reservoir is described in U.S. Pat. No. 5,788,980. Addition of fatty acid esters increases the solubility of estrogens (e.g., estradiol) and progestins. The increased solubility leads to a zero-order rate of delivery over a prolonged period of time.

Another approach was disclosed in U.S. Patent Application Publication No. 2014/0209100 which describes devices that include a reservoir of at least one vaginally delivered drug, wherein the reservoir is surrounded by a hydrophilic elastomer. Such devices are capable of releasing hydrophilic drugs at a substantially zero-order rate over extended period of times.

PCT Publication No. WO 2009/036999 describes a drug delivery system containing at least three different compartments/layers that releases different progestogen/estrogen combinations for contraception in a substantially constant ratio.

In summary, although a large number of device concepts have been described, all of them suffer from at least one of the following drawbacks: inability to adjust the release of multiple therapeutic components, difficult or expensive manufacturing process, inability to meet required release criteria to achieve the optimal targeted therapeutic effect and lack of stability upon storage and transport. This is especially true, when it comes to delivering highly polar or insoluble compounds like estriol.

Estriol has been used for the local therapy of certain menopausal symptoms. In US 2011/0086825 a topical formulation is described of progesterone, testosterone and estriol.

WO 2009/000954 describes the use of low dose estriol for the treatment/prevention of vaginal atrophy. US 2011/0312929 describes an estriol formulation with the capacity to self-limit the absorption of estriol for the treatment of urogenital atrophy, and in WO 2010/069621 the treatment of vaginal atrophy for women with a cardiovascular risk is described.

A film based estriol oral formulation for the buccal application of estriol is described in WO 2005/110358 by Elger et al. for the treatment of climacteric symptoms. The same group describes in U.S. Pat. No. 5,614,213 a transdermal product that releases estriol over 24 hours.

Estriol derivatives have been described. In U.S. Pat. No. 4,780,460, glycol esters of estriol have been described in order to form an aqueous crystalline suspension.

In U.S. Pat. No. 4,681,875, 3,17-estriol esters were disclosed for the prolonged subcutaneous application of estriol. Estriol esters were also disclosed in U.S. Pat. No. 6,894,038 for the treatment of autoimmune diseases such as multiple sclerosis.

It can be concluded that no approach has been described to generate long-lasting therapeutic plasma levels of estriol that would be needed in order to treat climacteric symptoms and to provide activity in the prevention of osteoporosis.

Another approach to overcome the hepatic estrogenicity problem of ethinyl estradiol is disclosed in WO 02/094278 describing the use of estetrol as the estrogenic component. Estetrol exhibits very weak estrogenic activity compared to estriol what leads to very high doses of 15 mg/day to reach pharmacological effects. Such high doses are prohibitive for the development of innovative drug delivery forms like patches and or vaginal rings.

For trimegestone, different applications have been described. In WO 03/084521 the use in combination with estradiol has been claimed as treatment for vasomotor symptoms. The contraceptive use as an oral formulation has been described in WO 01/37841 and EP 0917466. Special drug delivery options have been claimed for patches in WO/9747333 and FR 2749586 and in form of vaginal rings for the HRT indication in EP 0917466.

In summary it can be concluded that, to experts in the field, a contraceptive product that avoids the strong hepatic estrogenic effects of EE or those of estradiol at high oral doses is desirable.

### SUMMARY OF THE INVENTION

The objective of the present invention is to provide a non-oral product to effect hormonal contraception and to prevent/reduce intermenstrual bleeding and menopausal cycle disorders. In addition, the desired product should reduce the risks of venous thrombosis, as they occur on oral contraceptives.

According to the present invention, this objective is achieved by a vaginal ring that comprises (a) estriol or a precursor of this compound and (b) a progestogen or a precursor thereof, as well as pharmaceutical additives and/or excipients known per se from the state of the art.

The vaginal ring according to the present invention is applied for 21 to 28 days. The two compounds fixated on the vaginal ring are successively released.

According to the present invention, the progestogen is preferably one of the compounds trimegestone, gestodene, etonogestrel, levonorgestrel, drospirenone, or a precursor of any of those compounds.

The estriol or a precursor thereof is released at a daily dosage of preferably 0.05 mg to 1.0 mg, and progestogen or a precursor thereof is at a daily dosage of 0.05 mg to 0.5 mg.

A daily dosage of 0.3 mg of estriol or a precursor thereof and 0.1 mg of progestogen or a precursor thereof is particularly preferred. Where drospirenone is used as the progestogen, a daily dosage of 3 mg is preferred.

Most preferred are daily doses of 0.1 mg of estriol or a precursor thereof and 0.5 mg of progestogen or a precursor thereof.

In a preferred embodiment, the vaginal ring according to the present invention contains estriol and trimegestone. The two compounds are preferably released at daily dosages of 0.3 and 0.1 mg, respectively.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts the mean plasma concentration vs. time of estriol in different E3/TMG vaginal rings;

FIG. 2 depicts the mean plasma concentration vs. time of estriol in different E3/TMG vaginal rings;

FIG. 3 depicts the bleeding profiles during and after treatment using an E3/TMG vaginal ring;

FIG. 4 depicts the bleeding profiles during and after treatment using a different E3/TMG vaginal ring;

FIG. 5 depicts the individual serum concentrations of estradiol during single vaginal application of Device 3;

FIG. 6 depicts the individual serum concentrations of progesterone during single vaginal application of Device 3;

FIG. 7 depicts the means serum concentrations of estradiol during single vaginal application of Device 1 (Test 1), Device 2 (Test 2) and Device 3 (Test 3); and

FIG. 8 depicts the individual serum concentrations of progesterone during single vaginal application of Device 1 (Test 1), Device 2 (Test 2) and Device 3 (Test 3).

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

In an embodiment, the problems of the prior art are resolved by using estriol as estrogenic component and trimegestone as progestational component when delivered via a vaginal ring.

In an embodiment, an intravaginal drug delivery device (e.g., a vaginal ring) is composed of estriol and a progestin dispersed in a thermoplastic matrix. A variety of materials may be used as the thermoplastic matrix. Generally, the materials used in the intravaginal device are suitable for extended placement in the vaginal tract or the uterus. In an embodiment, a thermoplastic material used to form the intravaginal drug delivery device is nontoxic and non-absorbable in the subject. In other embodiments, the intravaginal drug delivery device may be formed from a biodegradable material. In some embodiments, the materials may be suitably shaped and have a flexibility allowing for intravaginal administration.

In an embodiment, an intravaginal drug delivery device is formed from an ethylene/vinyl acetate copolymer (EVA). A variety of grades may be used including grades having a low melt index, a high melt index, a low vinyl acetate content or a high vinyl acetate content. As used herein, EVA having a “low melt index” has a melt index of less than about 100 g/10 min as measured using ASTM test 1238. EVA having a “high melt index” has a melt index of greater than about 100 g/10 min as measured using ASTM test 1238. EVA having a “low vinyl acetate content” has a vinyl acetate content of less than about 20% by weight. EVA having a “high vinyl acetate content” has a vinyl acetate content of greater than about 20% by weight. The thermoplastic matrix of an intravaginal drug delivery device may be formed from EVA having a low melt index, a high melt index, a low vinyl acetate content or a high vinyl acetate content. In some embodiments, the thermoplastic matrix may include: mixtures of a low melt index and high melt index EVA or mixtures of low vinyl acetate content and high vinyl acetate content EVA.

The estriol and progestin may be dispersed in an EVA matrix to form an intravaginal drug delivery device. In an embodiment, an intravaginal drug delivery device consists essentially of estriol and a progestin dispersed in an EVA matrix. In an embodiment, an intravaginal drug delivery device consists of estriol and a progestin dispersed in an EVA matrix. In a preferred embodiment, an EVA having a high vinyl acetate content is used as a matrix for forming the intravaginal drug delivery device.

In some embodiments, the estriol and the progestin may be in separate compartments of the intravaginal drug delivery device. For example, an intravaginal drug delivery device may be composed of two or more compartments (e.g., segments) which are joined together. In an embodiment where the intravaginal drug delivery device is composed of multiple compartments, one or more of the compartments may include estriol while separate compartments may include the progestin. In a preferred embodiment, the intravaginal drug delivery device is composed of two compartments joined to each other. One of the two compartments is composed of estriol in an EVA matrix. The other of the two compartments is composed of a progestin in an EVA matrix. In one embodiment, the two or more compartments may be in the form of a cylinder. During formation, the ends of the cylindrical compartments are joined together to form a ring-shaped intravaginal drug delivery device.

In some embodiment, the intravaginal drug delivery device may be coated with an outer layer, referred herein as a skin. A “skin”, as used herein, is a layer of material that is substantially devoid of any estrogen or progestin materials at the time of manufacture. The phrase “substantially devoid of any estrogen or progestin material” means that there is less than 0.1% by weight of any estrogen or progestin material in the skin. In embodiments in which the intravaginal drug delivery device is composed of two or more compartments, each compartment may be coated with a skin or uncoated. In some embodiments, the estrogen may be disposed in an uncoated compartment and the progestin is disposed in a compartment coated with a skin. In some embodiments, the skin has a thickness of less than 1 mm, less than 500 μm, or less than 300 μm. By varying the thickness of the skin, the release rate of the active agent disposed in the core compartment may be controlled. In a preferred embodiment, the compartment and skin are composed of ethylene vinyl acetate. In a preferred embodiment, the compartment is composed of an ethylene vinyl acetate having a high vinyl acetate content, while the skin is composed of an ethylene vinyl acetate having a low vinyl acetate content.

In a preferred embodiment, the intravaginal drug delivery device is composed of two cylindrical segments joined together to form a ring. The first segment is composed of estriol dispersed in an ethylene vinyl acetate (EVA) matrix, where the EVA has a high vinyl acetate content. The second segment is composed of a progestin (preferably trimegestone) dispersed in an ethylene vinyl acetate (EVA) matrix, where the EVA has a high vinyl acetate content. The second segment is coated with a skin composed of EVA having a low vinyl acetate content and having a thickness of less than 500 μm.

In an embodiment, estriol is released at a daily dose between 0.05 and 1.0 mg and the progestin or a precursor thereof is released at a daily dose of 0.05 to 0.5 mg. In an embodiment, estriol is released at a daily dose between 0.15 and 0.5 mg and the progestin or a precursor thereof is released at a daily dose of 0.1 to 0.2 mg. In an embodiment, estriol is released in a daily dose of 0.3 mg and progestin or a precursor thereof is released at a daily dose of 0.1 mg. When the progestin is trimegestone, the estriol is released in a daily dose of 0.3 mg and trimegestone is released in a daily dose of 0.1 mg.

17α-ethinyl testosterone (Ethisterone) is the forerunner of several generations of progestins. Most of them have androgenic properties, potentially leading to an array of adverse effects such as decreased HDL-lipoproteins, disturbed carbohydrate metabolism and stimulatory effects on sebaceous glands and acne. In order to avoid these problems, new progestins with less androgenicity were developed (first-, second-, and third-generation progestins in the epidemiologic literature.

Cardiovascular health problems, including potentially fatal outcomes, such as pulmonary embolism, are the main concern with combined hormonal contraception. The involved pathologic events are apparently modulated by interactions of estrogen and progestin at tissue level, most importantly at the hepatic level. Androgenic properties of first- and second generation progestins partially blunt the adverse effects of EE on blood coagulation. The elimination of excessive hepatic effects of the estrogen in a contraceptive combination may require parallel inventive steps on the side of the progestin. Estriol (E3) as the estrogen and trimegestone (TMG) represent parts of the concept.

The cardiovascular benefit of second generation progestins in EE containing preparations, and the rationale to recommend their use in contraception, is based on their superior androgenic activity. Third generation progestins are less protective due to their reduced androgenic properties. The estrogen in a combination, therefore, determines the appropriate choice of the progestin. Improvements on the estrogen-side of the problem permits or demands a more physiologic non-androgenic substance on the progestin-side. This is an important aspect of this invention with regard to the non-androgenic TMG in combination with estriol (E3).

Treatment with a vaginal ring releasing a progestogen and estriol surprisingly achieves reliable inhibition of follicular maturation and ovulation with optimal cycle control. Here, the progestogen trimegestone was found to be particularly effective. Hepatic estrogen parameters within the normal range show the absence of hepatic estrogen effects at anti-ovulatory dosages superior cycle stabilisation by estriol in combination with a progestogen, preferably trimegestone, is surprising in view of negative experiences with estradiol regarding cycle control.

The invention describes a new approach to a combined hormonal contraceptive based on vaginal ring (VR-) technology and strategic options of this parenteral way of administration.

Follicle maturation and ovulation: Method using VR-technology revealed a safe inhibition of ovulation. This is based on the synergism of estrogen and progestin, namely estriol and trimegestone, suppressing both, growth of follicles as indicated by basal levels of estradiol after VR insertion and a delay of ovulation beyond 7 days after VR removal.

Cycle: Estradiol is metabolized to estrone which is much less estrogenic at endometrial tissue level in presence of progesterone. The resulting endometrial estrogen deficiency is causing bleeding disturbances. Contrary to estradiol, estriol is superior to the much more potent estradiol in the presence of a progestin in endometrial tissue. With a constant flow of estriol- and TMG- from a VR, a stable endometrial cycle and an almost complete absence of irregular uterine bleedings is achieved.

Hepatic effects of estriol and TMG using VR-administration: Absence of hepatic effects (e. g. unchanged SHBG as established surrogate parameter for hemostatic factors) as compared to EE or after high oral doses in mg/day-range of estradiol: No alteration of the array of estrogen- and androgen-modulated liver functions, among them vulnerable factors of the hemostatic system is expected.

Adverse metabolic effects vs second generation progestins' androgenicity. Adverse effects apply to lipid- and carbohydrate metabolism, carrier proteins, electrolyte metabolism and more) become avoidable. This applies also to androgenic effects on the skin: stimulation of sebaceous gland function, acne.

It is also surprising that, while ovulation is reliably inhibited at dosages within a defined range, there is no intermenstrual or withdrawal bleeding, respectively, during or after treatment. Administration according to the present invention thus makes it possible to apply a bleeding-free form of therapy over a period of three to even six menstrual cycles.

In addition to contraception, use of the combination according to the present invention by vaginal ring also allows treatment of disorders of ovarian hormone secretion and the bleeding pattern in the menstrual cycle. Relevant disturbances are very frequent in menopause. Use of the vaginal ring according to the present invention with the combination of progestogen and estriol compensates for existing or therapy-induced deficiencies of the ovarian hormones. This therapy maintains the menstrual cycle and protects the endometrium from the consequences of predominant estrogen effects, e.g. in the presence of anovulatory cycles and inadequate functioning of the corpus luteum.

At these low dosages, uterine bleeding was suppressed not only with the vaginal ring inserted, but withdrawal bleeding after removal of the ring also was. Withdrawal bleeding did not occur after a dosage of 0.4 mg of estriol and 0.05 mg of trimegestone per day. The same applies to the dosage of 0.3 mg of estriol together with 0.1 mg of trimegestone.

Blood loss during menstruation is a considerable and useless health burden for women. These combinations make it possible to reduce the frequency of bleeding during contraceptive or peri-menopausal treatment. Other positive therapeutic aspects of estriol/trimegestone treatment—the reliable control of ovarian hormone secretion, the metabolic substitution effect, and the absence of hepatic oestrogen parameter excursion to avoid a risk of thrombosis—are not affected by this variant.

Pregnancy is possible until menopause. It can be associated with considerable risks in increased age. Therefore, it is advantageous to choose a therapy for menopausal disorders that also has a contraceptive effect and prevents disorders of the cycle with irregular bleeding.

The present invention avoids known risks of venous thrombosis as they occur in case of conventional oral hormonal contraceptives or oral hormone preparations in menopause therapy.

This risk exists according to the state of the art with estradiol when administered orally and with ethinylestradiol regardless of the route of administration (concerns oral or transdermal therapy and administration by vaginal ring).

A role of the estrogen in a combined contraceptive is replacement of the estrogens that are secreted by maturing follicle and corpus luteum and fail upon inhibition of ovulation. This substitution affects the entire organism. A further role is the inhibition of gonadotropin secretion and thus of ovulation as a prerequisite for an anti-fertile effect. Ultimately, acceptance of the preparation is an important prerequisite, especially avoidance of intermenstrual bleeding. Problems with intermenstrual bleeding are often the reason for rejection or discontinuation of hormonal therapy.

The developments since the introduction of oral contraceptives document the limits of the efforts to develop the ideal contraceptive pill based on the currently still dominant ethinylestradiol technology. Dosage reductions beyond a critical limit lead to incomplete suppression of ovarian function with the risk of pregnancy and impaired bleeding control. In addition, the problem of increased thrombosis risks persists despite reduced dosage of ethinylestradiol. The threshold for metabolic effects of ethinylestradiol in the liver is far below the dosages required for contraceptive treatment.

Use of natural estrogens solves the problem of unwanted hepatic effects only gradually and at the expense of suboptimal cycle control. However, the androgenic properties of a progestogen reduce the risk of thrombosis only gradually. As a result, there is always a risk of thrombosis when estrogens are used in oral substitution therapy or oral contraception. The risk is dosage-dependent and increases with age.

The vaginal ring according to the present invention is capable of avoiding the major disadvantages of combined hormonal contraception. The improvements concern the contraceptive effects, cycle control, and tolerability.

In terms of tolerability, the hepatic effects of the estrogen component of a combination preparation are of particular importance. The risk of thromboembolism affects all combined contraceptives and dosage forms containing ethinylestradiol.

Oral use of estradiol requires very high dosages. This dosage is higher by a factor of approximately 40 (2.0 mg orally vs. 0.05 mg parenterally) than the parenteral dosage with the same effect. At an oral dosage of 2 mg of estradiol per day, significant hepatic estrogen effects and an increased risk of thromboembolism have been shown.

The present invention solves this problem by applying estriol via a vaginal ring in combination with a progestogen, preferably trimegestone: Hepatic estrogenicity is avoided. The combination claimed herein provides adequate substitution for the inhibited ovarian hormone secretion.

Unlike estradiol, estriol in humans does not cause the hepatic adverse effects known from ethinylestradiol and estradiol. The absence of this hepatic effect in the case of estriol also applies to extreme dosages and has been experimentally confirmed in a direct comparison with estradiol.

With regard to hepatic effects in humans, a qualitative difference between the natural estrogens estriol and estradiol may be presumed. Even upon therapeutic vaginal application of 1.0 mg estriol, no increase in SHBG (sexual hormone-binding globulin) was observed, as is impressively the case with ethinylestradiol. Estrogen-increased secretion is considered a surrogate parameter for the hepatic factors of the haemostasis system. A difference between the two natural estrogens, estradiol and estriol, is also proven, very surprisingly, in terms of cycle stabilization without intermenstrual bleeding with estriol under the influence of a progestogen.

The main reason why it has not been possible to develop a more widely accepted oral contraceptive based on estradiol is poor cycle control. Surprisingly, studies in human beings have now shown that estriol in combination with a progestogen, preferably trimegestone, can prevent intermenstrual bleeding. Unexpectedly, estriol is able to prevent intermenstrual bleeding better than is possible with the much more potent natural oestrogen estradiol.

With the combination of estriol and a progestogen, preferably trimegestone, on a vaginal ring according to the present invention, reliable inhibition of follicular maturation and ovulation as well as good cycle control are unexpectedly achieved at the same time. Estrogen-modulated liver functions are not affected.

In sheep, it was observed under experimental conditions that on trimegestone the estrogenic effect of estriol in the ovine genital organs is not inhibited. This is surprising, since progestogen generally weakens or eliminates the effect of estrogens (estradiol) in the genital tract. In contrast, the effect of estriol in the tissue of the genital tract is not abrogated by progestogens.

In the perimenopause, estradiol levels are significantly lowered despite the cycles being maintained. Increased secretion of FSH (follicle stimulating hormone) in the wake of a counter-regulation accelerates follicular maturation, reduces ovulatory cycles, and leads to dysfunctional bleeding in the aftermath of cycles with pathological or absent luteal phase.

Treatment with the vaginal ring according to the present invention compensates for existing or therapy-induced deficiencies in ovarian hormones. Applied in monthly intervals, the substitution maintains the menstrual cycle. Consequences of predominant estrogen effects in anovulatory cycles or insufficient function of the corpus luteum lead in the longer term to pathological remodelling processes in the uterus, which can ultimately lead to malignant degeneration, i.e. endometrial carcinoma. This unfavourable development can be prevented by treatment with the vaginal ring according to the present invention.

The low dosages of progestogen (preferably trimegestone) according to the present invention also make it possible to control the disturbed functions of the ovary without bleeding occurring. With several vaginal ring treatment cycles in a sequence, additionally the intervals between the bleedings can be increased. This is advantageous for clinical pictures in which menstrual complaints and other complaints/pains during menstruation play an important role, e.g. endometriosis.

The vaginal ring with the active ingredient combination of progestogen (preferably trimegestone) and estriol, according to the present invention, provides: (a) inhibition of follicular maturation/ovulation and ovarian hormone secretion; (b) peri-menopausal hormone replacement; (c) treatment of cycle disorders; (d) prevention of hyperplasia the endometrium and endometrial carcinoma as a consequence thereof; (e) reliable contraceptive effect in sexually active women; and (f) in contrast to conventional oral estrogen/progestogen therapy reduced or no risk of thrombosis.

Menopausal therapy with the vaginal ring according to the present invention has numerous advantages from the point of view of contraception as well. Pregnancy at an advanced age is associated with considerable risks. Hormonal contraceptives at an advanced age are cautioned against because of an increased risk of thrombosis and thromboembolism. With the vaginal ring according to the present invention, there is now an alternative available that provides contraception while eliminating the risk of thrombosis.

Clinical studies of the vaginal rings according to the present invention showed that, with ovulation reliably inhibited, bleeding did not occur during treatment or after removal of the vaginal ring according to the present invention when lower trimegestone dosages of up to 0.05-0.10 mg/day and estriol dosages of up to 0.3 mg/day were used. Preferably, successive use of several vaginal rings according to the present invention generates bleeding-free phase/cycles.

In case of endometriosis, use of the vaginal ring according to the present invention likewise provides considerable benefits. Endometriosis comprises disorders caused by the appearance of uterine mucosa outside the uterus. This pathological tissue is subject to cyclic changes due to ovarian hormone secretion. Severe pain conditions during the monthly bleeding are typical.

Use of the vaginal ring according to the present invention dampens ovarian hormone secretion, and the release of estriol and progestogen (preferably trimegestone) prevents intermenstrual bleeding and withdrawal bleeding. For example, it is advantageous to wear the vaginal ring according to the present invention successively, optionally without pauses, for 3×21 days or 3×28 days.

In addition to the advantages described, which distinguish estriol and, as progestogen, in particular trimegestone, high acceptance and low blood loss are to be mentioned. Accordingly, the vaginal rings according to the present invention can be optimized in terms of the release of estriol and progestogen with regard to a menopausal preparation or for the treatment of endometriosis.

### Pharmacological Aspects of the Invention

Ethinylestradiol is therapeutically used as an orally active estrogen. Its anti-ovulatory activity in conjunction with progestogens well known. Superior characteristics (vs. estradiol) are its cycle-stabilizing properties in the control of endometrial bleeding. Acceptance of a hormonal contraceptive depends on the success of the latter.

The problem with ethinylestradiol is its extremely strong hepatic estrogenicity, which involves secretion of a wide range of proteins, including transport proteins such as TBD, CBD, SHBG, angiotensinogen, lipoproteins and coagulation system factors.

Attempts to solve the problem of strong hepatic estrogenicity by dosage reduction have failed. Dosages far below the therapeutic threshold still trigger strong effects in the liver. In humans, ethinylestradiol is more effective by a factor of 4 to 16 in the liver in terms of FSH-inhibiting efficacy than the natural hormone estradiol is. The pronounced hepatic estrogenicity of ethinylestradiol is a property of the molecule. It occurs regardless of the route of application. Accordingly, the risk of thromboembolism cannot be reduced by parenteral application.

A dosage of 20 μg of ethinylestradiol is considered the lower limit for inhibition of ovulation and acceptable cycle control. However, this low dosage does not solve the problem of hepatic estrogenicity.

Experiments in sheep showed that the oestrogenic effects of estriol in the genital tract are not attenuated in the presence of progestogen/trimegestone. This estrogenic effect in the genital tract in the presence of a progestogen leads to prevention of spotting and intermenstrual bleeding in humans. Such bleedings on the “pill” are an expression of an endometrial estrogen deficiency. It is surprising that this is possible with estriol, whereas with the much more potent estradiol this effect does not occur.

In our studies of the inventive vaginal ring, treated women were in the follicular phase when the vaginal ring was inserted. It was possible to control the growth of follicles and to decrease the endogenous estrogen secretion, and block the ovulation. The used release rate of estriol alone decreased the levels of FSH and LH. Suppression of early follicle stages after estriol in combination with TMG indicates a strong synergism of both hormonal components. This finding suggests very high contraceptive efficacy for a E3/TMG vaginal ring. Furthermore, delay of ovulation after VR removal exceeds seven days, permitting a ring-free interval in the practical use.

Absence of bleedings and spotting in women using an E3/TMG vaginal ring uncovers a difference between ethinylestradiol and estriol on the one hand and estradiol on the other. Bleeding control with EE and E3 but not with E2 was observed. The observed properties make the E3/TMG vaginal ring an ideal candidate for treating cycle disorders and bleeding disorders in combination with providing contraceptive protection.

The claimed dosage of E3 in the E3/TMG vaginal ring is below the level which was used in reported exploratory studies with E3. When surrogate parameter SHBG was measured under E3 in women, no relevant changes were seen under the entire range of tested doses. The expectation is that the low dose and blood concentrations of E3 are below a level which may lead to responses as known for high oral doses of E2 or for any tested doses of EE. The E3/TMG vaginal ring may lead to a reduction of cardiovascular morbidity and mortality compared with conventional EE and progestin combinations using this E3/TMG vaginal ring as a combined hormonal contraceptive.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Vaginal Ring Production

### Example 1—Trimegestone, Reservoir Systems

### Premix Preparation:

Trimegestone loaded powder blends containing identical loadings in the core (=1.053%) are prepared by dry blending the active agent (trimegestone) and ethylene vinyl acetate (EVA 28) using different blending techniques (e.g., tumble blending, high shear blender) and blending parameters, yielding a powder blend where the active agent is homogeneously distributed in the blend.

### Co-Extrusion:

The trimegestone loaded ethylene vinyl acetate is co-extruded using a twin-screw extruder for the drug loaded core material and a single screw extruder for forming a drug free ethylene vinyl acetate skin layer over the core. The ethylene vinyl acetate skin is formed with a lower VA content (9%). The single screw extruder speed is adjusted to screw speed in order to yield the target skin thickness. By running the single screw extruder at low screw speeds of <5 rpm, a skin thickness of 135 μm can be achieved. Doubling the single screw extruder speed produces a skin thickness of 190 μm, and by a further screw speed increase of above 20 rpm, an increased skin thickness of 320 μm can be produced. The obtained co-extrudate is subsequently cooled to yield co-axial fibers with an outer diameter of 4.0 mm and the distinct skin thicknesses of 135 μm, 190 μm and 320 μm. The co-extrudate diameter and sphericity may be controlled in-line using a multiple laser head system

### Example 2—Estriol, Matrix System

### Premix Preparation:

Estriol loaded powder blends of 30% (w/w) are prepared by dry blending estriol and EVA 28 (ethylene vinyl acetate having a vinyl acetate content of 28%) using different blending techniques (e.g., tumble blending, active blending via high shear forces) and blending parameters, yielding a powder blend where the active agent is homogeneously distributed in the blend.

### Extrusion:

In a matrix extrusion step, the drug loaded premix is processed via hot melt extrusion using a twin-screw extruder and subsequent cooling at ambient temperature to yield drug loaded matrix strands of 4.0 mm outer diameter. The temperature configuration is slightly adapted depending on the drug loading and hence, the resulting melt viscosity to achieve a stable extrusion process and spherical extrudates.

### Example 3—Estriol/Trimegestone Vaginal Ring, Segmented (Matrix/Reservoir) System

Combining Estriol with Trimegestone Containing Segments and Ring Closure:

Estriol loaded segments, prepared according to Example 2, are cut into segments of 61 mm, 92 mm and 122 mm. Trimegestone containing co-extrudates having a skin of 190 μm, prepared according to Example 1, are cut into segments of 30, 60, and 90 mm. Cutting is done either manually or using a semi-automated system. The two segments are then joined in 2 subsequent welding steps (e.g., hot air welding, injection molding) inside a ring-shaped mold with single or multiple cavities to yield one or multiple vaginal rings of 4.0 mm cross-sectional diameter and 54 mm outer diameter. As welding material, the drug free ethylene vinyl acetate, serving as the carrier for the matrix and the core polymer for the reservoir system, is used.

Three test samples were prepared. Device 1 was produced by combining an estriol segment of 122 mm with a TMG segment of 30 mm. Device 2 was produced by combining an estriol segment of 92 mm with a TMG segment of 60 mm. Device 3 was produced by combining an estriol segment of 61 mm with a TMG segment of 90 mm.

### In Vitro Release Rates

For in vitro dissolution testing, a rotational incubator operated at 37±0.5° C. is used. The type of dissolution medium, its volume and the incubator rotational speed are selected to provide sink conditions. Samples of 1 mL are withdrawn every 24±0.5 h (and multiples thereof) over 21 or 28 days, the medium is replaced every 24±0.5 h (and multiples thereof) by fresh media, and the samples are analyzed for the drug content via (ultra) high performance liquid chromatography (UPLC/HPLC). Daily release rates of TMG and E3 of Device 2 and Device 3 are shown in Table 1

TABLE 1
Daily release rates of TMG and E3
Device 2
V2

| | 370 mg | | 6 mg |
|---|---|---|---|
| Day | E3 | Day | TMG |
| 1 | 2210 | 1 | 231 |
| 2 | 746 | 2 | 136 |
| 3 | 555 | 3 | 121 |
| 4 | 464 | 4 | 113 |
| 5 | 403 | 5 | 105 |
| 6 | 366 | 6 | 102 |
| 7 | 342 | 7 | 100 |
| 8 | 321 | 8 | 96 |
| 9 | 295 | 9 | 93 |
| 10 | 280 | 10 | 90 |
| 11 | 267 | 11 | 86 |
| 12 | 257 | 12 | 84 |
| 13 | 244 | 13 | 82 |
| 14 | 234 | 14 | 80 |
| 15 | 230 | 15 | 77 |
| 16 | 221 | 16 | 74 |
| 17 | 209 | 17 | 73 |
| 18 | 202 | 18 | 70 |
| 19 | 199 | 19 | 73 |
| 20 | 193 | 20 | 70 |
| 21 | 194 | 21 | 71 |
| Average | 311 | | 90 |

Daily release rates of TMG and E3
Device 3
V3

| | 250 mg | | 9 mg |
|---|---|---|---|
| Day | E3 | Day | TMG |
| 1 | 1435 | 1 | 347 |
| 2 | 496 | 2 | 210 |
| 3 | 371 | 3 | 185 |
| 4 | 306 | 4 | 174 |
| 5 | 268 | 5 | 164 |
| 6 | 245 | 6 | 158 |
| 7 | 228 | 7 | 153 |
| 8 | 215 | 8 | 148 |
| 9 | 198 | 9 | 140 |
| 10 | 189 | 10 | 137 |
| 11 | 180 | 11 | 133 |
| 12 | 172 | 12 | 129 |
| 13 | 165 | 13 | 125 |
| 14 | 159 | 14 | 122 |
| 15 | 156 | 15 | 118 |
| 16 | 148 | 16 | 113 |
| 17 | 141 | 17 | 111 |
| 18 | 136 | 18 | 107 |
| 19 | 135 | 19 | 108 |
| 20 | 131 | 20 | 105 |
| 21 | 132 | 21 | 106 |
| Average | 209 | | 137 |

### Clinical Studies to Determine Ovulation-Inhibiting Effects and to Record Bleeding Patterns

Testing of three vaginal ring prototypes (Device 1, Device 2, and Device 3, described above) in 12 women as preformed. The expected average release rate per day of each device is described in Table 2. Vaginal ring (VR) insertion was performed after pill cycle completion and menstrual bleeding. Observation of a VR over a period of 21 days. Follow-up after removal of the VR. All VR variants tested inhibit ovulation. Ovulation inhibition persisted for more than 7 days after removal of the rings. Signs of follicular maturation at the start of treatment (higher oestradiol levels) disappear during treatment. The effect reflects inhibition of follicular maturation. For the most part, there were no bleeding events during treatment at all and, where they did occur, only to a very small extent (“spotting”). Withdrawal bleeding was observed only after the highest trimegestone dosage.

TABLE 2
Tested VR variant overview

| | Estriol (mg/day) | Trimegestone (mg/day) |
|---|---|---|
| Device 1 | 0.400 | 0.050 |
| Device 2 | 0.300 | 0.095 |
| Device 3 | 0.209 | 0.137 |

### Clinical Investigations

### Mean Plasma Concentration of Estriol and Trimegestone

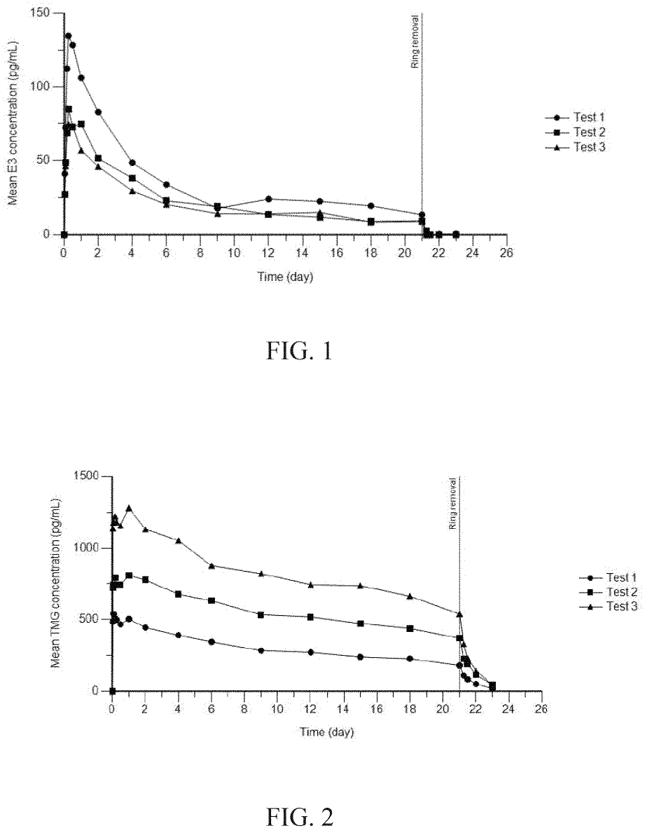

FIG. 1 depicts the mean plasma concentration (pg/mL) vs. time of estriol in Device 1, Device 2 and Device 3 after vaginal insertion in 12 women over 21 days. FIG. 2 depicts the mean plasma concentration (pg/mL) vs. time of trimegestone in Device 1, Device 2 and Device 3 after vaginal insertion in 12 women over 21 days.

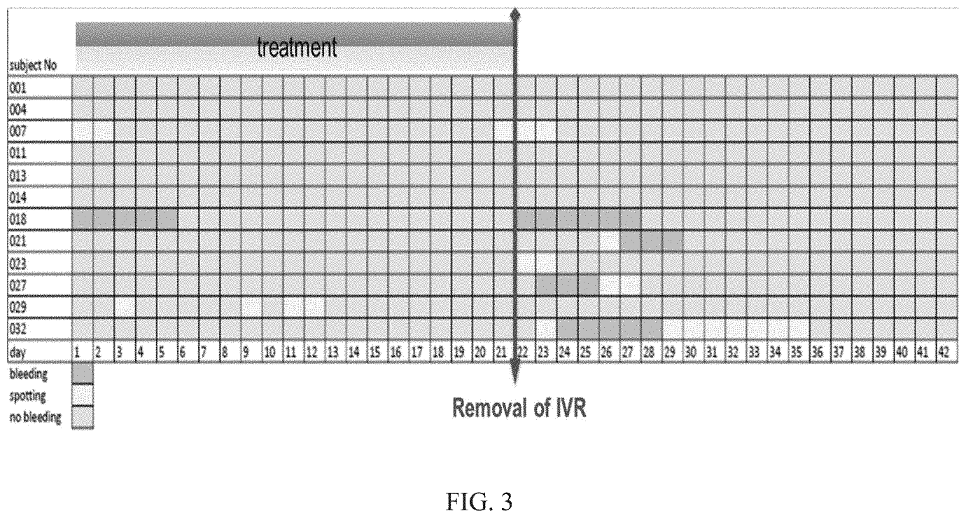

FIG. 3 depicts the bleeding profiles during and after treatment using Device 3 with a drug release of 0.209 mg of estriol/day and 0.137 mg of trimegestone/day mg. With the vaginal ring, bleedings are singular, predominantly insignificant events. Withdrawal bleeding can be detected after removal of the ring.

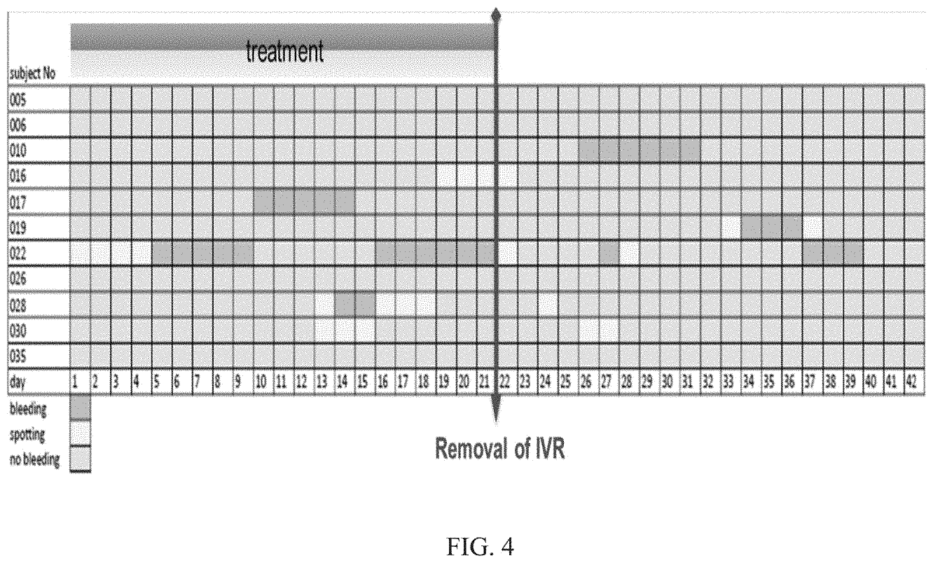

FIG. 4 depicts the bleeding profiles during and after treatment using Device 2 with a drug release of about 0.3 mg of estriol/day and about 0.1 mg of trimegestone/day. In most cases, no bleeding was observed before and after withdrawal of the device.

### Clinical Study Ovulation Inhibition

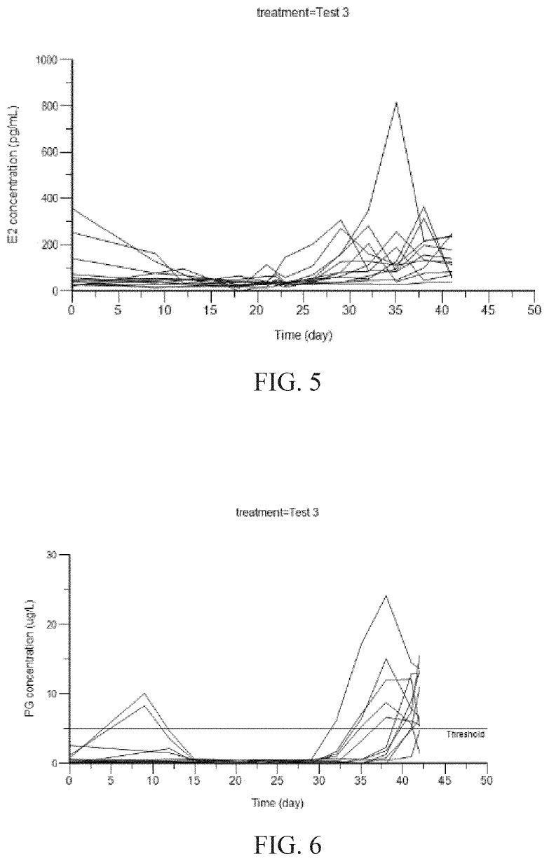

The course of the estradiol levels in the circulation reflects follicular maturation in the ovary. FIG. 5 depicts the individual serum concentrations of estradiol during single vaginal application of Device 3. Device 3 reduces values elevated after a pill break and keeps them very low for the duration of the treatment. After removal of the vaginal ring, the estradiol values increase starting from baseline. This increase reflects follicular maturation. This increase from zero and the latency of ovulations of 9-15 days demonstrates deep suppression of follicular maturation in the ovary with Device 3. FIG. 6 depicts the individual serum concentrations of progesterone during single vaginal application of Device 3. The point at which the progesterone values rise from zero marks the time of ovulation.

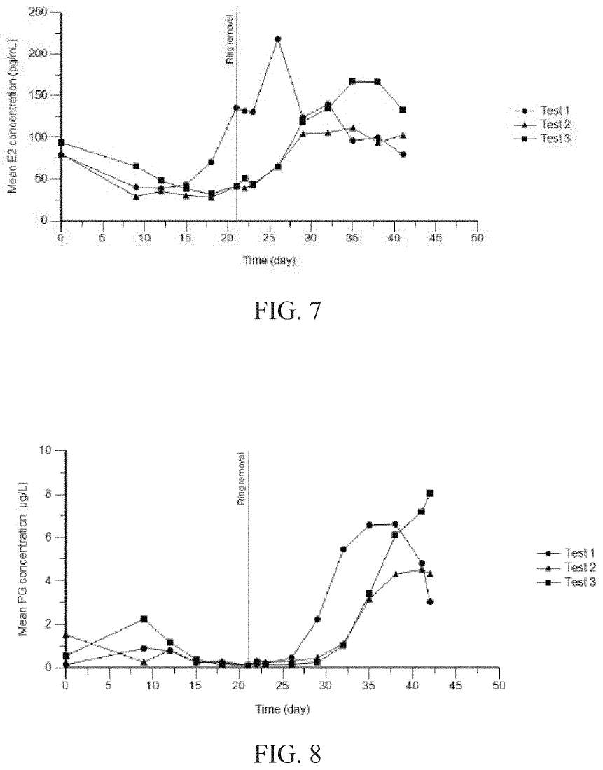

FIG. 7 depicts the means serum concentrations of estradiol during single vaginal application of Device 1 (Test 1), Device 2 (Test 2) and Device 3 (Test 3). FIG. 8 depicts the individual serum concentrations of progesterone during single vaginal application of Device 1 (Test 1), Device 2 (Test 2) and Device 3 (Test 3). The point at which the progesterone values rise from zero marks the time of ovulation.

There can be no doubt about the reliable contraceptive effect of the tested devices. Apart from occasional “spotting” there was little or no metrorrhagia or intermenstrual bleeding with the tested devices and no withdrawal bleeding after removal of the devices.

### Clinical Testing for Influencing Haemostasis Parameter

The influence on haemostasis factors can be tested in an open label study in young and healthy women over a period of 7 cycles by comparing the test compound with Nuvaring. The primary variables selected as sensitive activation markers for coagulation status are the absolute changes in prothrombin fragments 1 and 2, D-dimer, and the (pro)coagulatory parameters (fibrinogen, Factor II, Factor VII, and Factor VIII activity) and anti-coagulatory parameters (antithrombin III, protein C, protein S) and APC resistance.

The advantages of the vaginal ring according to the present invention can be summarised as follows. Formation of a combined contraceptive based on estriol as the estrogen component. Formation of an estriol/trimegestone vaginal ring for combined contraception. The E3/TMG vaginal rings exhibit reliable and persistent inhibition of ovulation, surprising because of the use of a “weak” estrogen (estriol). Contraceptive applications using an E3/TMG vaginal ring allows breaks in monthly intervals to be possible. The E3/TMG vaginal rings exhibit inhibited ovulation, but bleeding-free VR cycles at low trimegestone dosages. Serial E3/TMG vaginal ring cycles can be used for extended bleeding-free cycle phases (HRT, perimenopause, endometriosis). Bleeding control using estriol, unlike estradiol, is not subject to degradation in the target organ under progestogen dominance (experiment on sheep, evidence through very good cycle control in humans). Bleeding under progestogen dominance, in the case of estradiol, are expressions of local estrogen deficiency due to degradation of the estradiol in the human uterus. Estriol is not subject to this local degradation, no degradation=no local oestrogen deficiency=no bleeding. Implications for the selection of the progestogen. Non-androgenic progestogens beneficial for combination with estriol, e.g. trimegestone. Separate progestogen application and vaginal ring therapy at the end of a longer bleeding-free vaginal ring series. Menopause—Implications of omission of hepatic risk by use of estriol. This makes it possible to use an E3/TMG vaginal ring for peri-menopausal hormonal therapy.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An intravaginal drug delivery device comprising estriol in combination with a progestin or a precursor thereof; wherein the intravaginal drug delivery device releases estriol and the progestin, over a period of at least 21 days, in an amount sufficient to:
(a) produce a contraceptive state in a female subject; and
(b) inhibit or prevent menstrual bleeding during and after use of the intravaginal drug delivery device.

2. The device of claim 1, wherein the device is composed of two or more compartments, wherein one of the two or more compartments comprises estriol and a separate one of the two or more compartments comprises the progestin.

3. The device of claim 2, wherein the compartment comprising estriol is an uncoated compartment comprising estriol in a matrix comprising ethylene vinyl acetate.

4. The device of claim 3, wherein the matrix comprises ethylene vinyl acetate having a high vinyl acetate content.

5. The device of claim 2, wherein the compartment comprising the progestin is a coated compartment comprising the progestin in a core matrix comprising ethylene vinyl acetate and a skin layer comprising ethylene vinyl acetate substantially devoid of any estrogens or progestins.

6. The device of claim 5, wherein the core matrix comprises ethylene vinyl acetate having a high vinyl acetate content and wherein the skin comprises ethylene vinyl acetate having a low vinyl acetate content.

7. The device of claim 2, wherein the two or more compartments are in the form of a cylinder, and wherein the cylindrical compartments are joined together to form a ring.

8. The device of claim 1, wherein the device releases estriol in doses between 0.05 and 1.0 mg/day.

9. The device of claim 1, wherein the progestin is a non-androgenic progestin selected from the group consisting of trimegestone, gestodene, etonogestrel, levonorgestrel, drospirenone, or a precursor of any of those compounds.

10. The device of claim 1, wherein the progestin is trimegestone.

11. The device of claim 10, wherein the device releases trimegestone in doses between 0.05 and 0.5 mg/day.

12. The device of claim 1, wherein the device has a substantially annular form.

13. The device of claim 1, wherein the device releases estriol and the progestin, over a period of at least 21 days, in an amount sufficient to not produce venous thrombosis in the female subject during treatment.

14. The device of claim 1, wherein the device releases estriol and the progestin, over a period of at least 21 days, in an amount sufficient to not produce changes in haemostasis parameters in the female subject during treatment.

15. The device of claim 1, wherein the device releases estriol and the progestin, over a period of at least 21 days, in an amount such that an unchanged level of sex hormone binding globulin is observed in the female subject during treatment.

16. A method of producing a contraceptive state in a subject comprising positioning an intravaginal drug delivery device in the vagina or uterus of a female, wherein the intravaginal drug delivery device comprises estriol in combination with a progestin or a precursor thereof; wherein the intravaginal drug delivery device releases estriol and the progestin, over a period of at least 21 days, in an amount sufficient to: (a) produce a contraceptive state in a female subject and (b) inhibit or prevent menstrual bleeding during and after use of the intravaginal drug delivery device.

17. A method of producing a contraceptive state in a subject comprising positioning an intravaginal drug delivery device in the vagina or uterus of a female, wherein the intravaginal drug delivery device comprises estriol in combination with a progestin or a precursor thereof; wherein the intravaginal drug delivery device releases estriol and the progestin, over a period of at least 21 days, in an amount sufficient to: (a) produce a contraceptive state in a female subject and (b) inhibit or prevent menstrual bleeding during and after use of the intravaginal drug delivery device, wherein the treatment also simultaneously reduces bleeding disorders, climacteric cycle irregularities, and venous thrombosis.

18. The method of claim 17, wherein the treatment has no substantial impact on haemostasis parameters in the female subject during treatment.

19. An intravaginal drug delivery device comprising estriol in combination with a progestin or a precursor thereof; wherein the intravaginal drug delivery device releases estriol and the progestin, over a period of at least 21 days, in an amount sufficient to:
(a) provide peri-menopausal hormone replacement in a female subject; and
(b) inhibit or prevent bleeding disorders and/or climacteric cycle irregularities during and after use of the intravaginal drug delivery device.

20. An intravaginal drug delivery device comprising estriol in combination with a progestin or a precursor thereof; wherein the intravaginal drug delivery device releases estriol and the progestin, over a period of at least 21 days, in an amount sufficient to prevent endometrial hyperplasia and endometrial carcinoma.
